(19) United States
(12) Patent Application Publication
(10) Pub. No.: US 2025/0326756 A1
(43) Pub. Date: Oct. 23, 2025

(54) METHOD FOR PRODUCING 3,6-DISUBSTITUTED-IMIDAZO[1,2-B]PYRIDAZINE COMPOUNDS

(71) Applicant: NUVATION BIO INC., New York, NY (US)

(72) Inventors: Fenger Zhou, Hangzhou (CN); Doug Dagang Chen, Hangzhou (CN); Fei Guo, Hangzhou (CN); Zengrong Ren, Tianjin (CN); Lijie Shang, Tianjin (CN); Huidong Wang, Tianjin (CN)

(21) Appl. No.: 18/866,777

(22) PCT Filed: May 18, 2022
(86) PCT No.: PCT/CN2022/093570

§ 371 Date: Nov. 18, 2024

(51) Int. Cl. C07D 487/04; A61K 31/5025

(52) U.S. Cl. CPC C07D 487/04; A61K 31/5025

(57) ABSTRACT

Provided are methods for producing 3,6-disubstituted-imidazo[1,2-b]pyridazine compounds or the salts thereof.

### TECHNICAL FIELD

This disclosure relates to methods for producing 3,6-disubstituted-imidazo[1,2-b]pyridazine compounds or a salt thereof.

### BACKGROUND

3-{4-[(2R)-2-Aminopropoxy]phenyl}-N-[(1R)-1-(3-fluorophenyl)ethyl]-imidazo[1,2-b]pyridazin-6-amine is a known ROSI receptor tyrosine kinase inhibitor and neurotrophic tyrosine receptor kinase (NTRK) inhibitor, and has the following chemical structure:

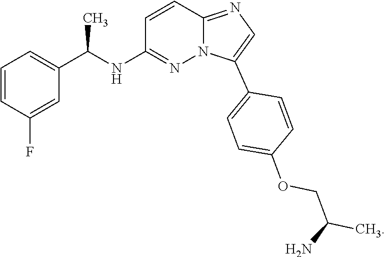 [SMILES: C[C@@H](N)COc1ccc(-c2cnc3ccc(N[C@H](C)c4cccc(F)c4)nn23)cc1]

It is known that 3-{4-[(2R)-2-aminopropoxy]phenyl}-N-[(1R)-1-(3-fluorophenyl)ethyl]-imidazo[1,2-b]pyridazin-6-amine is useful for the treatment of cancers.

### SUMMARY

This disclosure is based on the unexpected discovery that a salt (e.g., a phosphate salt) of 3,6-disubstituted-imidazo[1,2-b]pyridazine compound can be used to prepare 3-{4-[(2R)-2-aminopropoxy]phenyl}-N-[(1R)-1-(3-fluorophenyl)ethyl]-imidazo[1,2-b]pyridazin-6-amine in an improved manufacturing method (e.g., having a significantly improved yield).

In one aspect, this disclosure features a manufacturing method that includes reacting a compound of formula (2):

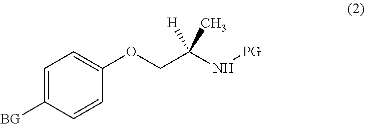

with a salt of a compound of formula (3):

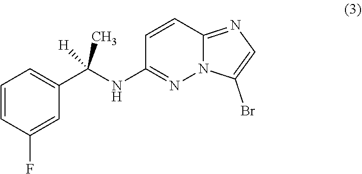 [SMILES: C[C@@H](Nc1ccc2ncc(Br)n2n1)c1cccc(F)c1]

in the presence of a palladium catalyst, a base, and a solvent to form a compound of formula (4):

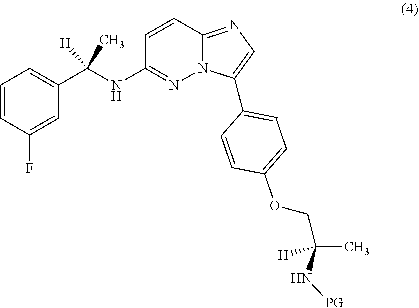

in which BG is a boron-containing group (e.g., a boronic ester or boronic acid group), and PG is a protecting group for a nitrogen atom. In some embodiments, the above method can further include removing the protecting group PG from the compound of formula (4) to form a compound of formula (5):

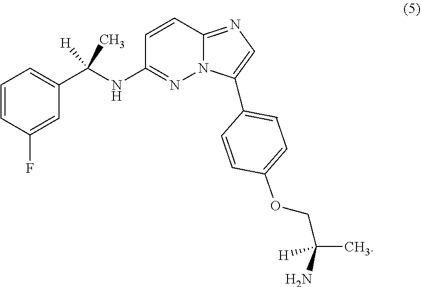 [SMILES: Cc1cc(OC[C@@H](C)N)ccc1-c1cnc2ccc(N[C@H](C)c3cccc(F)c3)nn12]

In another aspect, this disclosure features a pharmaceutical composition that includes particles comprising 3-{4-[(2R)-2-aminopropoxy]phenyl}-N-[(1R)-1-(3-fluorophenyl)ethyl]-imidazo[1,2-b]pyridazin-6-amine monoadipate, and a pharmaceutically acceptable carrier, in which the particles have a particle size D50 of from about 20 μm and 70 μm.

Other features, objects, and advantages will be apparent from the description and the claims.

### DETAILED DESCRIPTION

This disclosure generally relates to methods of producing 3-{4-[(2R)-2-aminopropoxy]phenyl}-N-[(1R)-1-(3-fluorophenyl)ethyl]-imidazo[1,2-b]pyridazin-6-amine (i.e., the compound of formula (5)) and its salts.

In one aspect, this disclosure features manufacturing methods that include reacting a compound of formula (2):

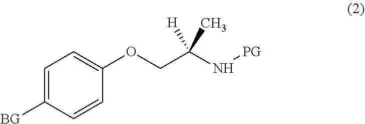

with a salt of a compound of formula (3) (i.e., (R)-3-bromo-N-(1-(3-fluorophenyl)ethyl)imidazo[1,2-b]pyridazin-6-amine):

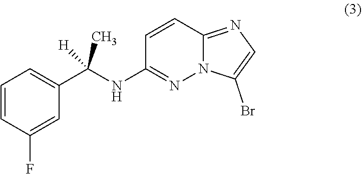 [SMILES: C[C@@H](Nc1ccc2ncc(Br)n2n1)c1cccc(F)c1]

in the presence of a palladium catalyst, a base, and a solvent to form a compound of formula (4):

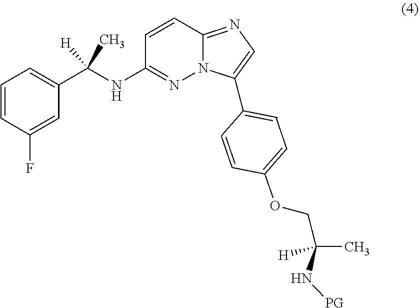

in which BG is a boron-containing group (e.g., a boronic ester or boronic acid group), and PG is a protecting group for a nitrogen atom. In some embodiments, the reaction above is a Suzuki coupling reaction.

In some embodiments, the salt of the compound of formula (3) can be an organic or inorganic salt, which can be obtained by reacting the compound of formula (3) with an organic or inorganic acid. Examples of suitable salts of the compound of formula (3) include a phosphate salt, a chloride salt, a sulfate salt, a fumarate salt, a citrate salt, a tartrate salt, an oxalate salt, a succinate salt, a 2,5-dihydroxybenzoate salt, an adipate salt, a p-toluenesulfonate salt, or a malate salt. Preferably, a phosphate salt of the compound of formula (3) is used in the methods described herein. In some embodiments, the phosphate salt of the compound of formula (3) can include from at least about 1.35 molar (e.g., at least about 1.4 molar, at least about 1.45 molar, or at least about 1.5 molar) to at most about 1.65 molar (e.g., at most about 1.6 molar, at most about 1.55 molar, or at most about 1.5 molar) of phosphoric acid per 1 molar of the compound of formula (3). In some embodiments, the phosphate salt of the compound of formula (3) can include about 1.5 molar of phosphoric acid per 1 molar of the compound of formula (3). Without wishing to be bound by theory, it is believed that, because the salt (e.g., a phosphate salt) of the compound of formula (3) is in a solid, crystalline form, it can be easily isolated from its preparation reaction in a high purity and in a high yield. In addition, without wishing to be bound by theory, it is believed that using a salt (e.g., a phosphate salt) of the compound of formula (3) as a starting material can result in the compound of formula (4) in a significantly higher yield than using the compound of formula (3) free base (e.g., in a liquid form) as a starting material.

In some embodiments, the amount of the compound of formula (2) can range from at least about 0.8 molar (e.g., at least about 0.85 molar, at least about 0.9 molar, at least about 0.95 molar, or at least about 1 molar) to at most about 1.2 molar (e.g., at most about 1.15 molar, at most about 1.1 molar, at most about 1.05 molar, or at most about 1 molar) per 1 molar of the salt of the compound of formula (3). In some embodiments, the molar ratio of the compound of formula (2) over the salt of the compound of formula (3) is about 1.1:1.

The protecting group (PG) for the nitrogen atom described herein is not particularly limited as long as it is a substituent that reduces the reactivity of the nitrogen atom to an electrophilic addition reaction. For example, the protecting groups disclosed in Protective Groups in Organic Synthesis (T. W. Green and P. G. M. Wuts, John Wiley & Sons, Inc., New York, 1991) can be used. In some embodiments, the protecting group is a tert-butoxycarbonyl group, a fluorenylmethoxycarbonyl group, or a benzyloxycarbonyl group.

In some embodiments, BG is boron-containing group suitable for Suzuki coupling reaction. Examples of suitable BG include boronic ester groups

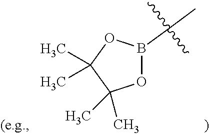 [SMILES: *B1OC(C)(C)C(C)(C)O1]

or boronic acid groups

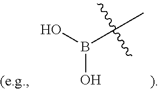 [SMILES: *B(O)O]

In some embodiments, the palladium catalyst described herein is a divalent palladium catalyst or a zero-valent palladium catalyst. An example of a zero-valent palladium catalyst is [tris(2-methylphenyl)phosphine]palladium (0).

In some embodiments, the palladium catalyst described herein includes a reaction product of a monodentate phosphine or a bidentate phosphine with a palladium compound. Examples of suitable monodentate phosphines include triphenylphosphine, tri-t-butylphosphine, and tris(2-methylphenyl)phosphine. Examples of suitable bidentate phosphines include 1,1-bis(diphenylphosphino)methane and 1,2-bis(diphenylphosphino)ethane. Examples of suitable palladium compounds include palladium chloride and palladium acetate. In some embodiments, the palladium catalyst described herein can include a reaction product of palladium acetate and triphenylphosphine. Without wishing to be bound by theory, it is believed that using a reaction product of palladium acetate and triphenylphosphine as a catalyst in the reaction between the compound of formula (2) and a salt of the compound of formula (3) can be advantageous over a conventional catalyst (e.g., [1,1′-bis(diphenylphosphino)ferrocene]dichloro-palladium (II)-dichloromethane (Pd(dppf)Cl₂·CH₂Cl₂) because the former catalyst can be readily removed from the reaction and a much smaller amount of the former catalyst is needed to complete the reaction to produce a product with a higher yield, which would improve the reaction efficiency and reduce product costs.

In some embodiments, the reaction between a compound of formula (2) and a salt of the compound of formula (3) can be carried out using a relatively small amount of a palladium catalyst. In some embodiments, the amount of the palladium catalyst used in this reaction can range from at least about 0.1 mol % (e.g., at least about 0.2 mol %, at least about 0.4 mol %, at least about 0.5 mol %, at least about 0.6 mol %, at least about 0.8 mol %, or at least about 1 mol %) to at most about 5 mol % (e.g., at most about 4.5 mol %, at most about 4 mol %, at most about 3.5 mol %, at most about 3 mol %, at most about 2.5 mol %, at most about 2 mol %, at most about 1.5 mol %, or at most about 1 mol %) per 1 molar of the compound of the formula (3). Without wishing to be bound by theory, it is believed that using a salt of the compound of formula (3) (which is a solid) as a starting material can significantly reduce the amount of the palladium catalyst used to obtain the compound of formula (4) compared to using the compound of formula (3) free base (e.g., in a liquid form) as a starting material, which would substantially reduce the cost of manufacturing the final product (i.e., the compound of formula (5) or a salt thereof).

In some embodiments, the base for use in the above reaction can be any suitable base that facilitates a Suzuki coupling reaction. Examples of suitable bases include potassium hydroxide, sodium hydroxide, sodium carbonate, potassium carbonate, and cesium carbonate.

In general, the solvent that can be used in the above reaction is not particularly limited. In some embodiments, the solvent does not inhibit the aromatic substitution reaction involving a C—H activation reaction catalyzed by palladium. Examples of suitable solvent include dimethylacetamide (DMAc), dimethylformamide (DMF), N-methyl-2-pyrrolidone (NMP), dimethyl sulfoxide (DMSO), 4-dioxane, and diethylene glycol dimethyl ether. In some embodiments, the solvent is miscible with water.

In some embodiments, the methods described herein can further include removing the protecting group PG from the compound of formula (4) to form a compound of formula (5):

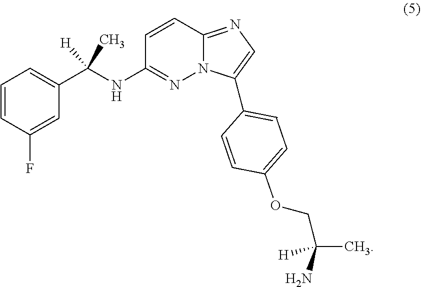 [SMILES: Cc1cc(OC[C@@H](C)N)ccc1-c1cnc2ccc(N[C@H](C)c3cccc(F)c3)nn12]

In some embodiments, the deprotection reaction can be performed in the presence of a mineral acid (e.g., hydrochloric acid). In some embodiments, the compound of formula (5) can be isolated by addition of a base (e.g., sodium hydroxide) to the solution (e.g., a HCl solution) obtained from the above reaction to adjust the pH to a suitable value (e.g., about 12) to allow the compound of formula (5) to crystalize and precipitate from the solution. Without wishing to be bound by theory, it is believed that using a mineral acid in the deprotection reaction and using a base to precipitate the compound of formula (5) from the reaction solution involves easy handling and can significantly increase the yield (e.g., from about 72% to about 90%) compared to a conventional method of using an organic acid (e.g., trifluoroacetic acid) in the deprotection reaction and isolating the compound of formula (5) by column chromatography.

In some embodiments, the above method can further include reacting the compound of formula (5) with an acid (e.g., an adipic acid) to form a salt (e.g., an adipate salt) of the compound of formula (5). Examples of suitable salt include inorganic acid salts and organic acid salts (e.g., amino acid salts). Examples of suitable inorganic salts include hydrohalides (e.g., a hydrofluoride, hydrochloride, hydrobromide, or hydroiodide salt), a nitrate, a perchlorate, a sulfate, and a phosphate. Examples of suitable organic acid salts include C₁-C₆ alkylsulfonates (e.g., a methanesulfonate, a trifluoromethanesulfonate, or an ethanesulfonate), arylsulfonates (e.g., a benzenesulfonate or a p-toluenesulfonate), an acetate, a malate, a fumarate, a succinate, a citrate, an ascorbate, a tartrate, an oxalate, and an adipate. Examples of suitable amino acid salts include a glycine salt, a lysine salt, an arginine salt, an ornithine salt, a glutamate salt, and an aspartate salt.

In some embodiments, the methods described herein can further include milling (e.g., wet milling) the salt of the compound formula (5) to form particles with a suitable size. The milling can be performed by methods known in the art. In some embodiments, the particles obtained from the milling and containing the salt (e.g., the monoadipate salt) of the compound of formula (5) can have a median particle size D50 of from at least about 20 μm (e.g., at least about 25 μm, at least about 30 μm, at least about 35 μm, or at least about 40 μm) to at most about 70 μm (e.g., at most about 65 μm, at most about 60 μm, at most about 55 μm, at most about 50 μm, or at most about 45 μm). Without wishing to be bound by theory, it is believed that, if the particle size of is too large (e.g., having a D50 larger than 70 μm), the particles containing the salt of the compound of formula (5) may have a undesirable dissolution profile (e.g., the particles can have a dissolution rate too low to meet regulatory requirements). Further, without wishing to be bound by theory, it is believed that, if the particle size of is too small (e.g., having a D50 smaller than 20 μm), the production yield of the salt of the compound of formula (5) may be too low.

In some embodiments, particles containing the salt (e.g., the monoadipate salt) of the compound of formula (5) can have a particle size D90 of from at least about 50 μm (e.g., at least about 60 μm, at least about 70 μm, at least about 80 μm, at least about 90 μm, or at least about 100 μm) to at most about 150 μm (e.g., at most about 140 μm, at most about 130 μm, at most about 120 μm, at most about 110 μm, or at most about 100 μm). In some embodiments, particles containing the salt (e.g., the monoadipate salt) of the compound of formula (5) can have a particle size D10 of from at least about 1 μm (e.g., at least about 1.5 μm, at least about 2 μm, at least about 4 μm, at least about 5 μm, at least about 6 μm, at least about 8 μm, at least about 10 μm, at least about 12 μm, or at least about 14 μm) to at most about 25 μm (e.g., at most about 24 μm, at most about 22 μm, at most about 20 μm, at most about 18 μm, at most about 16 μm, at most about 14 μm, at most about 12 μm, or at most about 10 μm). Without wishing to be bound by theory, it is believed that, when particles described herein have a relatively low D90 and a relatively high D10, the particles would have an improved particle size uniformity.

In some embodiments, the methods described herein can further include reacting a compound of formula (1):

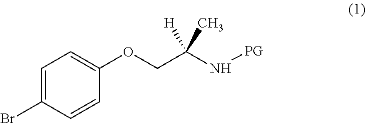

with a boron-containing agent (e.g., bis(pinacolato)diboron) to form the compound of formula (2). In some embodiments, this reaction can be performed in the presence of a palladium catalyst (e.g., the reaction product of palladium acetate and triphenylphosphine), a base (e.g., potassium acetate), and a solvent (e.g., a solvent described herein such as DMAc). Without wishing to be bound by theory, it is believed that using the reaction product of palladium acetate and triphenylphosphine as a catalyst in this reaction can be advantageous over a conventional catalyst (e.g., [1,1′-bis(diphenylphosphino)ferrocene]dichloro-palladium (II)-dichloromethane (Pd(dppf)Cl₂·CH₂Cl)) because (1) the reaction product of palladium acetate and triphenylphosphine can be readily removed from the reaction and (2) the reaction product of palladium acetate and triphenylphosphine can improve the yield of this reaction even when a much smaller amount of palladium acetate is used, which would improve the reaction efficiency and reduce product costs.

In some embodiments, the reaction between the compound of formula (1) and a boron-containing agent (e.g., bis(pinacolato)diboron) can be performed at a relatively high temperature. For example, the reaction can be performed at a temperature of from at least about 85° C. (e.g., at least about 90° C., at least about 95° C., or at least about 100° C.) to at most about 120° C. (e.g., at most about 115° C., at most about 110° C., at most about 105° C.). Without wishing to be bound by theory, it is believed that performing the reaction between the compound of formula (1) and a boron-containing agent within the above reaction temperature range can significantly shorten the reaction time (e.g., from 12 hours to 2 hours) and improve the reaction yield (e.g., from about 75% to about 100%) compared to performing the reaction at a conventional temperature (i.e., 80° C.).

In some embodiments, the methods described herein can further include reacting 1-bromo-4-fluorobenzene

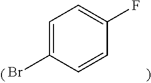

with D-alaninol

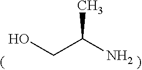

to form (R)-1-(4-bromophenoxy)propan-2-amine

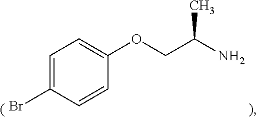

and protecting the amino group in (R)-1-(4-bromophenoxy)propan-2-amine (e.g., by reacting (R)-1-(4-bromophenoxy)propan-2-amine with di-tert-butyl dicarbonate (Boc₂O)) to form the compound of formula (1).

In some embodiments, the methods described herein can further include reacting a compound of formula (6) (i.e., 3-bromo-6-chloroimidazo[1,2-b]pyridazine):

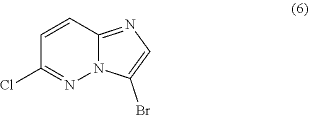 [SMILES: Clc1ccc2ncc(Br)n2n1]

with a compound of formula (7) (i.e., (R)-1-(3-fluorophenyl) ethan-1-amine);

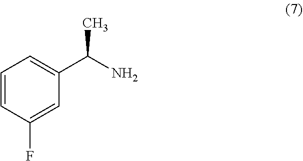

to form the compound of formula (3). In some embodiments, this reaction can be performed in the presence of a base (e.g., cesium fluoride). In some embodiments, the methods described herein can further include reacting the compound of formula (3) with an acid (e.g., phosphoric acid) to form an acid additional salt (e.g., a phosphate salt) of the compound of formula (3). Examples of suitable acids include phosphoric acid, hydrochloric acid, sulfuric acid, fumaric acid, citric acid, tartaric acid, oxalic acid, succinic acid, 2,5-dihydroxybenzoic acid, adipic acid, p-toluenesulfonic acid, or malic acid. Without wish to be bound by theory, it is believed that performing the reaction between the compound of formula (6) and the compound of formula (7) in the presence of cesium fluoride can significantly shorten reaction time (e.g., from 20-24 hours to 13-15 hours) and reduce reaction temperature (e.g., from about 130° C. to about 110-120° C.) compared to performing the reaction in the presence of a conventional baes (e.g., potassium fluoride). In addition, without wishing to be bound by theory, it is believed that forming an acid addition salt of the compound of formula (3) in the manufacturing methods described herein is advantageous over forming the compound of formula (3) free base at least because (a) an acid addition salt of the compound of formula (3) can be readily isolated from the reaction mixture in a solid form with a high purity (which would satisfy the Regulatory Starting Material (RSM) requirements imposed by regulators (e.g., the Food and Drug Administration (FDA) and European Medicines Agency (EMA)), while the compound of formula (3) free base generally cannot be isolated in a solid form (which would fail the RSM requirements) and (b) an acid addition salt of the compound of formula (3) can be isolated in a higher yield than the compound of formula (3) free base.

In some embodiments, the amount of the compound of formula (6) is larger than and in excess of the amount of the compound of formula (7) in the above reaction. For example, the molar ratio of the compound of formula (6) over the compound of formula (7) is at least about 1:02:1 (e.g., at least about 1.04:1, at least about 1.05:1, at least about 1.06:1, at least about 1.08:1, at least about 1.1:1, or at least about 1.15:1) or at most about 1.2:1. Without wishing to be bound by theory, it is believed that using the compound of formula (6) in an excess amount can significantly improve the purity of the product obtained from the above reaction at least because the compound of formula (6) is easier to be removed from the reaction product than the compound of formula (7).

In some embodiments, this disclosure features a pharmaceutical composition that includes particles containing a salt (e.g., a pharmaceutically acceptable salt) of the compound of formula (5) (e.g., 3-{4-[(2R)-2-aminopropoxy]phenyl}-N-[(1R)-1-(3-fluorophenyl)ethyl]-imidazo[1,2-b]pyridazin-6-amine monoadipate) and a pharmaceutically acceptable carrier. In some embodiments, the particles can be obtained by using the milling method described herein. In some embodiments, the particles thus obtained can have a suitable particle size, such as those described above. For example, the particles thus obtained can have a particle size D50 of from at least about 20 μm to at most about 70 μm, a particle size D90 of from at least about 50 μm to at most about 150 μm, and/or a particle size D10 of from at least about 1 μm to at most about 25 μm.

Without wishing to be bound by theory, it is believed that the pharmaceutical composition containing particles having the particle size described herein (e.g., having a particle size D50 of from at least about 20 μm to at most about 70 μm) can have a dissolution rate meeting regulatory requirements. For example, the pharmaceutical composition described herein can have a dissolution amount of from at least about 75 wt % (e.g., at least about 80 wt %, at least about 82 wt %, at least about 84 wt %, at least about 85 wt %, at least about 86 wt %, at least about 88 wt %, at least about 90 wt %, at least about 92 wt %, at least about 94 wt %, at least about 95 wt %, at least about 96 wt %, at least about 98 wt %, or at least about 99 wt %) to about 100 wt % of the total weight of the active ingredient (e.g., the compound of formula (5)) in 45 minutes in an acetic acid dissolution medium having a pH of about 4 as measured by the method described in Example 6 below.

Examples of suitable pharmaceutically acceptable salts include acid addition salts, e.g., salts formed by reaction between the compound of formula (5) and hydrohalogen acids (such as hydrochloric acid or hydrobromic acid), mineral acids (such as sulfuric acid, phosphoric acid and nitric acid), and aliphatic, alicyclic, aromatic or heterocyclic sulfonic or carboxylic acids (such as formic acid, acetic acid, propionic acid, succinic acid, adipic acid, glycolic acid, lactic acid, malic acid, tartaric acid, citric acid, benzoic acid, ascorbic acid, maleic acid, hydroxymaleic acid, pyruvic acid, p-hydroxybenzoic acid, embonic acid, methanesulphonic acid, ethanesulphonic acid, hydroxyethanesulphonic acid, halobenzenesulphonic acid, trifluoroacetic acid, trifluoromethanesulphonic acid, toluenesulphonic acid, and naphthalenesulphonic acid).

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical carriers for delivery of the compound of formula (5) or its salts described herein. Examples of other carriers include colloidal silicon oxide, magnesium stearate, cellulose, sodium lauryl sulfate, and D&C Yellow #10.

The pharmaceutical composition described herein can optionally include at least one further additive selected from a disintegrating agent, binder, lubricant, flavoring agent, preservative, colorant and any mixture thereof. Examples of such and other additives can be found in “Handbook of Pharmaceutical Excipients”; Ed. A. H. Kibbe, 3rd Ed., American Pharmaceutical Association, USA and Pharmaceutical Press UK, 2000.

The pharmaceutical composition described herein can be adapted for oral administration or for administration via the respiratory tract (e.g., in the form of an aerosol or an air-suspended fine powder) to a subject in need of treatment of a disease (e.g., a cancer such as non-small cell lung cancer or thyroid cancer). In some embodiments, the composition can be in the form of tablets, capsules, powders, microparticles, and granules.

The pharmaceutical composition described herein generally includes a therapeutically effective amount of the compound of formula (5) or a salt thereof. “A therapeutically effective amount” refers to the amount of the pharmaceutical composition that is required to confer a therapeutic effect (e.g., reversing, alleviating, delaying the onset of, or inhibiting the progress of, a cancer or one or more symptoms thereof) on the treated subject.

The following examples are illustrative and not intended to be limiting.

### EXAMPLES

### Example 1: Preparation tert-butyl (R)-(1-(4-(4,4,5,5-tetramethyl-1,3,2-dioxaborolan-2-yl)phenoxy)propan-2-yl)carbamate (a Compound of Formula (2))

### Synthesis of tert-butyl (R)-(1-(4-bromophenoxy)propan-2-yl)carbamate (the Compound of Formula (1))

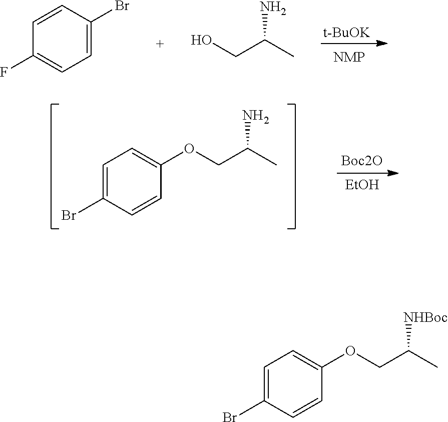

NMP (4.5-5.5×), potassium t-butyloxide (1.5 molar eq.), D-1-amino-2-propanol (1.1 molar eq.) were added to a reactor. After the mixture was stirred for 15-30 minutes, 1-bromo-4-fluorobenzene (1 molar eq.) was added. After the mixture was slowly heated to 65-75° C., it was stirred for 2-3 hours at this temperature. After in-process control (IPC) was qualified (i.e., the amount of limiting reagent 1-bromo-4-fluorobenzene was no more than 1% based on the relevant peak area in HPLC), the mixture was cooled down to 20-30° C. Isopropyl acetate (4.2-5.5×) and water (9.5-10.5×) were sequentially added to the above mixture. The mixture thus obtained was filtered by diatomaceous earth and the organic and aqueous phases were separated. After the aqueous phase was extracted with isopropyl acetate (4.5-5.5×), the organic phases were combined and washed with water (4.5-5.5×) twice. After the organic phase was concentrated to 3V, isopropyl acetate was replaced by ethanol twice by adding ethanol (8×) into the organic phase and concentrated. The organic phase was eventually concentrated to 3V. After THF (1.9-2.1×) was added, di-tert-butyl dicarbonate (1.3 molar eq.) was slowly added to the mixture at 0-10° C. The mixture was allowed to warm to 20-25° C. slowly and was stirred for 2-3 hours at this temperature. After IPC was qualified, ethanol (1.8-2×) was added, followed by addition of water (7-9×) slowly. The mixture was stirred for another 4-8 hours at this temperature. The mixture was then centrifuged and the filtered product was washed with an ethanol aqueous solution. The product thus obtained was recrystallized in an ethanol aqueous solution and dried to give the title compound with a yield of 69-75%. The product had a chiral purity of ≥99.90%, an individual impurity≤0.10%, total impurities≤0.50%, and residual water≤0.50%.

### Synthesis of tert-butyl (R)-(1-(4-(4,4,5,5-tetramethyl-1,3,2-dioxaborolan-2-yl)phenoxy)propan-2-yl)carbamate (a Compound of Formula (2))

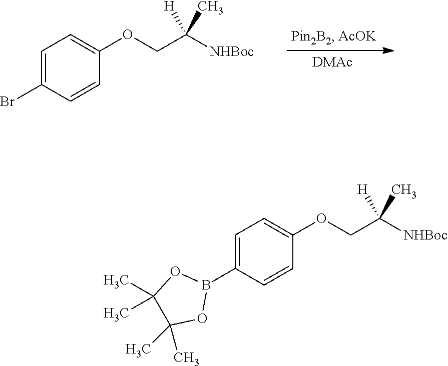

Tert-butyl (R)-(1-(4-(4,4,5,5-tetramethyl-1,3,2-dioxaborolan-2-yl)phenoxy)propan-2-yl)carbamate (i.e., a compound of formula (2) described herein) was prepared using a conventional method and an inventive method as follows:

### Conventional Method

Tert-butyl (R)-(1-(4-(4,4,5,5-tetramethyl-1,3,2-dioxaborolan-2-yl)phenoxy)propan-2-yl)carbamate was prepared following the procedures described in step 2 of Reference Example 1 in columns 27-28 of U.S. Pat. No. 9,187,489. Specifically, 10V of 1, 4-dioxane, 1.0 molar eq. of tert-butyl (R)-(1-(4-bromophenoxy)propan-2-yl)carbamate obtained above, 1.2 molar eq. of bis(pinacolato)diboron, 3.0 molar eq. of potassium acetate, and 0.1 molar eq. of Pd(dppf)Cl₂·CH₂Cl₂ were sequentially added into a four-neck flask. As used herein, IV refers 1 liter of a solvent per 1 Kg of a limiting reagent (which is tert-butyl (R)-(1-(4-bromophenoxy)propan-2-yl)carbamate in this reaction). The reaction mixture was deaerated for 20 minutes, heated to 80° C. and maintain under stirring at this temperature for 4 hours. After IPC was qualified (i.e., the amount of limiting reagent tert-butyl (R)-(1-(4-bromophenoxy)propan-2-yl)carbamate was no more than 1% based on the relevant peak area in HPLC), the reaction mixture was cooled down to 20° C. After 5V of ethyl acetate was added to the reaction mixture, the mixture was stirred for 10 minutes, and filtered to remove any insoluble matter. The mother liquor obtained from the filtration was concentrated, dried, and purified by silica column chromatography to obtain the title product. The NMR of the product thus obtained was consistent with the NMR of the product obtained in Reference Example 15 shown on pages 31-32 of U.S. Pat. No. 9,187,489.

### Inventive Method

2.85 Kg/Kg of N,N-Dimethylacetamide, 1.0 molar eq. of tert-butyl (R)-(1-(4-bromophenoxy)propan-2-yl)carbamate (i.e., the compound of formula (1) described above), 1.2 molar eq. of bis(pinacolato)diboron, and 0.006 molar eq. of triphenylphosphine were sequentially charged into a four-neck flask. The flask was then purged with nitrogen until the oxygen content was no more than 0.1%. After 2.0 molar eq. of potassium acetate and 0.002 molar eq. of palladium acetate were added into the reaction mixture, the flask was purge with nitrogen again and the oxygen content was monitored until it was no more than 0.1%. The reaction mixture was heated to 100° C. and maintained under stirring for 1 hour. After IPC was qualified, the reaction mixture was cooled to 20° C. After 5.93 Kg/Kg methyl tert-butyl ether and a sodium chloride solution were sequentially added to the reaction mixture, the mixture was stirred and allowed to stand to form two phases. After the upper organic phase was separated, the organic phase was washed by a sodium bicarbonate solution (i.e., by adding the sodium bicarbonate to the organic phase, stirring the mixture, and separating the organic phase). Palladium acetate was subsequently removed from the organic phase through a membrane stack. The organic phase was then sequentially washed by (1) 5 Kg/Kg of purified water and 0.09 Kg/Kg of ethylenediamine, (2) a sodium carbonate solution, and (3) a sodium chloride solution. The organic phase was then concentrated under reduced pressure to 10-20V at a temperature of no more than 40° C. After 6.8 Kg/Kg n-heptane was added to the organic phase, the organic phase was concentrated under reduced pressure to 60˜80V at a temperature of no more than 40° C. After 4.76 Kg/Kg n-heptane was added to the organic phase, the organic phase was concentrated under reduced pressure to 60˜80V at a temperature of no more than 40° C. The organic phase thus obtained was sampled and the residual methyl tert-butyl ether was measured to be no more than 1.0%. The solution thus obtained was heated to 45˜55° C. and stirred under that temperature. The solution was then cooled down to 10˜25° C. and stirred under that temperature until a solid was precipitated. After 2.04 Kg/Kg n-heptane was into the mixture, the mixture was cooled down to a temperature no more than −5° C. and maintain at that temperature for crystallization. The mixture was then filtered at a temperature of no more than −5° C. The solid thus obtained was dried in an oven at 15˜30° C. until the residual n-heptane is no more than 5%. ¹H NMR (400 MHz, DMSO-d₆) δ 7.62-7.53 (m, 2H), 6.96-6.81 (m, 3H), 3.90 (p, J=4.7, 4.2 Hz, 1H), 3.81 (tt, J=11.2, 5.9 Hz, 2H), 1.38 (s, 9H), 1.27 (s, 12H), 1.11 (d, J=6.2 Hz, 3H).

The experimental conditions and results of the conventional and inventive methods described above are summarized in Tables 1 and 2 below.

TABLE 1
Comparison of Conditions Between Conventional and Inventive Methods

| | | | | Compound | Bis(pina | | |
|---|---|---|---|---|---|---|---|
| Synthetic | | Reaction | | of Formula | colato)- | Catalyst and | Reaction |
| route | Solvent | Temp. | AcOK | (1) | diboron | Equivalent Amount | Time |
| Conventional | 1,4- | 80° C. | 3.0 | 1.0 | 1.2 | Pd(dppf)Cl₂•CH₂Cl₂ | 4 h |
| Method | Dioxane | | molar eq. | molar eq. | molar eq. | 0.1 molar eq. | |
| Inventive | DMAC | 100° C. | 2.0 | 1.0 | 1.2 | Triphenylphosphine | 1 h |
| Method | | | molar eq. | molar eq. | molar eq. | 0.006 molar eq. | |
| | | | | | | Palladium acetate | |
| | | | | | | 0.002 molar eq. | |

TABLE 2
Comparison of Experimental Results Between
Conventional and Inventive Methods

| Test item | Conventional Method | Inventive Method |
|---|---|---|
| Description | White solid | White solid |
| Purity | 99% | 99% |
| Yield | 74% | 88% |

As shown in Tables 1 and 2 above, the inventive method surprisingly resulted in a product with a yield much higher than the conventional method even though a much smaller amount of a palladium catalyst was used. Without wishing to be bound by theory, it is believed that (1) using solution extraction as a purification process in the inventive method can significantly improve the yield of this reaction compared to using column chromatography as a purification process in the conventional method; (2) using the reaction product of palladium acetate and triphenylphosphine as a catalyst in the inventive method can also improve the yield of this reaction compared to using Pd(dppf)Cl₂·CH₂Cl₂ as a catalyst in the conventional method; and (3) using Pd(dppf)Cl₂·CH₂Cl₂ in the conventional method is not cost-effective because a large amount (i.e., 0.1 molar eq.) of this catalyst is required, while only a small amount (i.e., 0.002 molar eq.) of palladium acetate is needed for the inventive method.

In addition, without wising to be bound by theory, it is believed that the column chromatography used in the conventional method is a relatively tedious process, lowers the yield of the final product, and is not suitable for commercial manufacturing. By contrast, although there are extra steps for removing the palladium catalyst in the inventive method, the solution extraction and recrystallization procedures used in the inventive method are relatively simple and can result in a product with a relatively high and consistent yield. In addition, unlike column chromatography, the solution extraction and recrystallization procedures used in the inventive method are readily scalable and suitable for commercial production.

### Example 2: Preparation (R)-3-bromo-N-(1-(3-fluorophenyl)ethyl)imidazo[1,2-b]pyridazin-6-amine (the compound of formula (3)) free base and phosphate salt

(R)-3-bromo-N-(1-(3-fluorophenyl)ethyl)imidazo[1,2-b]pyridazin-6-amine (i.e., the compound of formula (3) described herein) free base and phosphate salt were respectively prepared using a conventional method and an inventive method as follows:

### Conventional Method

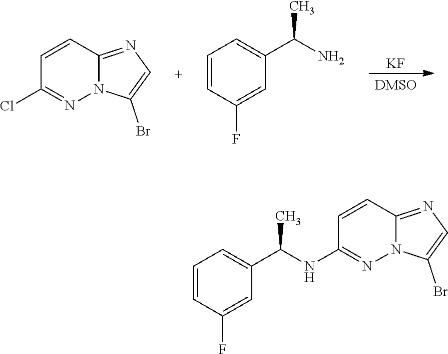

(R)-3-bromo-N-(1-(3-fluorophenyl)ethyl)imidazo[1,2-b]pyridazin-6-amine free base was prepared following the procedures described in step 1 of Example 1 in column 97 of U.S. Pat. No. 9,187,489. Specifically, 20V of dimethyl sulfoxide (DMSO), 1.0 molar eq. 3-bromo-6-chloroimidazo[1,2-b]pyridazine (i.e., the compound of formula (6) described above), 1.2 molar eq. of (R)-1-(3-fluorophenyl) ethan-1-amine (i.e., the compound of formula (7) described above), and 2.4 molar eq. of potassium fluoride were sequentially added to a four-neck flask. The reaction mixture was heated to 130° C. and maintained under stirring for 24 hours. The reaction mixture was sampled and, after IPC was qualified, cooled down to 20° C. The reaction mixture was then added dropwise into ice water (100V) and stirred for 1˜2 hours. The resulting solid was collected by filtration and was dissolved in hot ethyl acetate. The insoluble matter was removed by filtration under heating. The mother liquor thus obtained was concentrated to form a precipitate. The mixture was then filtered and the resulting solid was dried to give the compound of formula (3) free base as a brown low quality solid. The NMR of the compound thus obtained was consistent with the NMR of the product obtained from Step 1 in Example 21 of U.S. Pat. No. 9,187,489.

### Inventive Method

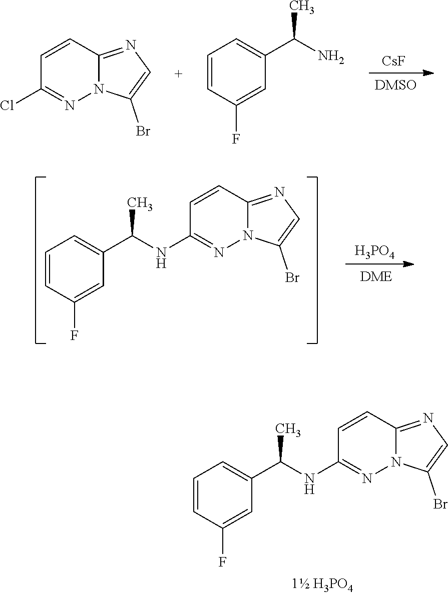

10V of DMSO, 1.1 molar eq. of 3-bromo-6-chloroimidazo[1,2-b]pyridazine (i.e., the compound of formula (6) described above), 1.0 molar eq. of (R)-1-(3-fluorophenyl) ethan-1-amine (i.e., the compound of formula (7) described above), 2.1 molar eq. of cesium fluoride were sequentially added to a four-neck flask. The reaction mixture was heated to 110˜120° C. and maintained under stirring for 13 hours. The reaction mixture was sampled and, after IPC was qualified, cooled down to 20° C. Subsequently, 5V of a 2N potassium hydroxide solution, 10V of toluene, and IV of acetonitrile were added to the reaction mixture obtained above. The mixture was stirred and allowed to form two phases, an aqueous phase and an organic phase. The aqueous phase was extracted with 5V of toluene and the toluene phase was combined with the previous organic phase. After 0.2 g/g activated charcoal was added into the organic phase, the mixture thus obtained was stirred for 1˜3 hours, and filtered under pressure, and the resulting solid was washed with toluene. The organic phase (which includes both the molar liquor from the filtration and the toluene solution) was washed with 5V of purified water until it had a pH of 7˜8 and concentrated to a volume of 3V.

After 18V of dimethoxyethane was added to the concentrated solution obtained above, the mixture thus obtained was heated to 45˜55° C. 2 Molar eq. of a dimethoxyethane phosphate solution was then added to the mixture. The reaction mixture thus obtained was stirred under 45˜55° C. for 1˜2 hours, and then cooled down 15˜25° C. The reaction mixture was filtered and dried to give the compound of formula (3) phosphate salt as a white solid. ¹H NMR (400 MHz, DMSO-d₆) δ 8.89 (s, 5H), 7.71 (d, J=9.7 Hz, 2H), 7.46 (s, 1H), 7.40-7.15 (m, 3H), 7.02 (dddd, J=9.1, 8.0, 2.7, 1.1 Hz, 1H), 6.76 (d, J=9.7 Hz, 1H), 4.95 (t, J=6.6 Hz, 1H), 1.49 (d, J=7.0 Hz, 3H).

The experimental conditions and results of the conventional and inventive methods described above are summarized in Tables 3 and 4 below.

TABLE 3
Comparison of Conditions Between Conventional and Inventive Methods

| | | | | | Compound | Compound | |
|---|---|---|---|---|---|---|---|
| | | | | | of Formula | of Formula | |
| Synthetic | | | Reaction | | (6) Molar | (7) Molar | Reaction |
| Route | Solvent | Base | Temp. | | Equivalents | Equivalents | Time |
| Conventional | DMSO | KF | 130° | C. | 1.1 molar eq. | 1.0 molar eq. | 24 h |
| Method | | | | | | | |
| Inventive | DMSO | CsF | 110~120° | C. | 1.0 molar eq. | 1.2 molar eq. | 13 h |

Method

TABLE 4
Comparison of Experimental Results Between
Conventional and Inventive Methods

| Test item | Conventional Method | Inventive Method |
|---|---|---|
| Description | Brown low quality solid | White solid |
| Purity | 96% | 99% |
| Yield | 87% | 89% |

As shown in Tables 3 and 4 above, the inventive method surprisingly resulted in a product with a higher purity and in a higher yield than the conventional method even though the inventive method was performed at a lower reaction temperature and in a shorter reaction time than the conventional method. Without wishing to be bound by theory, it is believed that, because the phosphate salt of the compound of formula (3) is in a solid, crystalline form, it can be easily isolated from its preparation reaction in a high purity and in a high yield.

In addition, the convention method resulted in a brown low quality solid product containing residual solvents and impurities. As this product (i.e., the compound of formula (3)) is a starting material for a drug (the compound of formula (5) or a salt thereof), it needs to be a stable physical solid under the RSM requirements. Since the conventional method did not produce a high quality solid product, it is not suitable for commercial manufacturing.

### Example 3: Preparation tert-butyl ((R)-1-(4-(6-(((R)-1-(3-fluorophenyl)ethyl)amino)-imidazo[1,2-b]pyridazin-3-yl)phenoxy)propan-2-yl)carbamate (a compound of formula (4))

Tert-butyl ((R)-1-(4-(6-(((R)-1-(3-fluorophenyl)ethyl)amino)imidazo[1,2-b]pyridazin-3-yl)phenoxy)propan-2-yl)carbamate (i.e., a compound of formula (4) described herein) was prepared using a conventional method and an inventive method as follows:

### Conventional Method

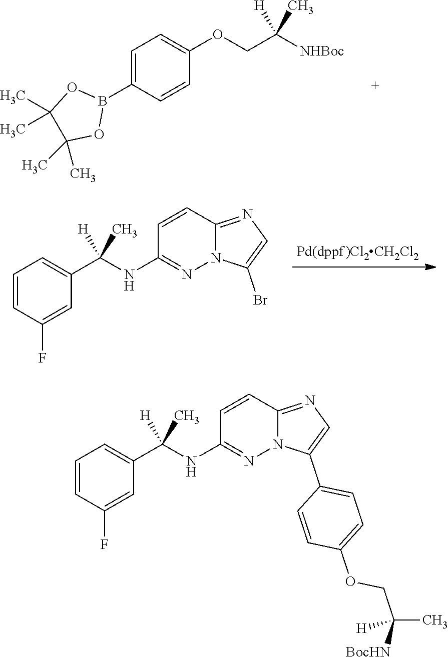 [SMILES: C[C@H](COc1ccc(B2OC(C)(C)C(C)(C)O2)cc1)NC(=O)OC(C)(C)C ; C[C@@H](Nc1ccc2ncc(Br)n2n1)c1cccc(F)c1 ; C[C@H](COc1ccc(-c2cnc3ccc(N[C@H](C)c4cccc(F)c4)nn23)cc1)NC(=O)OC(C)(C)C]

Tert-butyl ((R)-1-(4-(6-(((R)-1-(3-fluorophenyl)ethyl)amino)imidazo[1,2-b]pyridazin-3-yl)phenoxy)propan-2-yl)carbamate was prepared following the procedures described in step 2 of Example 21 in column 125 of U.S. Pat. No. 9,187,489. Specifically, 73.5 V of 1, 4-dioxane, 14.7 V of purified water, 1.0 molar eq. of the compound of formula (3) free base obtained in Example 2 above, 1.2 molar eq. of the compound of formula (2) obtained in Example 1 above, 4.0 molar eq. of potassium carbonate, and 0.1 molar eq. of Pd(dppf)Cl₂·CH₂Cl₂ were added into a four-neck flask. The reaction mixture was purged using nitrogen for 20 minutes, heated to 85° C., and stirred at this temperature for 1.5 hours. After IPC was qualified, the reaction mixture was cooled down to 20° C. After ethyl acetate and purified water were added into the reaction mixture, the mixture was stirred and allowed to form two phases. The organic phase was washed with a saturated sodium chloride solution, dried with anhydrous magnesium sulfate, concentrated, and dried. The crude product was purified by column chromatography to give the title compound. The NMR of the compound thus obtained was consistent with the NMR of the product obtained from Step 2 in Example 21 in column 125 of U.S. Pat. No. 9,187,489.

### Inventive Method

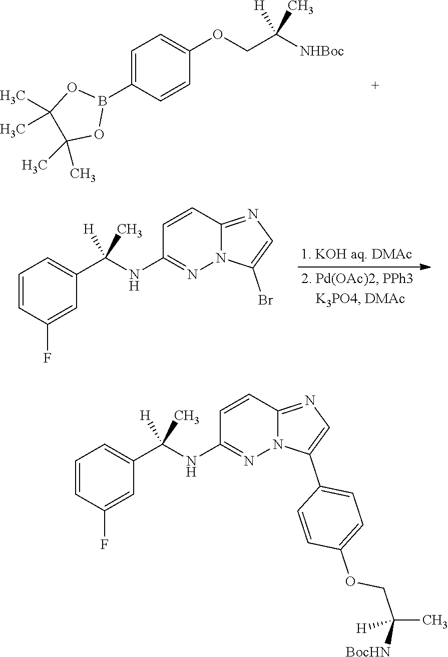 [SMILES: C[C@H](COc1ccc(B2OC(C)(C)C(C)(C)O2)cc1)NC(=O)OC(C)(C)C ; C[C@@H](Nc1ccc2ncc(Br)n2n1)c1cccc(F)c1 ; C[C@H](COc1ccc(-c2cnc3ccc(N[C@H](C)c4cccc(F)c4)nn23)cc1)NC(=O)OC(C)(C)C]

4.68 Kg/Kg of N, N-dimethylacetamide (DMAc), 1.0 molar eq. of the compound of formula (3)phosphate salt obtained in Example 2 above, 1.51 V of purified water, and 4.6 molar eq. of potassium hydroxide were sequentially added into a four-neck flask. At a temperature of 20˜30° C., 2.5 V of purified water, 1.5 molar eq. of potassium phosphate, and 1.1 molar eq. of the compound of formula (2) obtained in Example 1 above were added into the reaction mixture, which was stirred to completely dissolve the reagents. The reaction mixture was then purged with nitrogen until the oxygen content was no more than 0.1%. After 0.008 molar eq. of triphenylphosphine and 0.004 molar eq. of palladium acetate were added, the reaction mixture was purged again with nitrogen until the oxygen content was no more than 0.1%. The reaction mixture was then heated to 90° C. and stirred at this temperature for 3 hours. After IPC was qualified, the reaction mixture was cooled down to 20° C. After 4 Kg/Kg of purified water was added to the reaction mixture, the mixture was extracted by adding 9 Kg/Kg of ethyl acetate, stirred, allowed to stand and separate into two phases. The upper organic phase was sequentially washed with (1) 3 Kg/Kg of purified water and (2) a sodium bicarbonate solution. At the temperature of 20˜30° C., after 0.1 Kg/Kg of activated charcoal was added to the organic phase, the organic phase was stirred and filtered and the palladium content was measured. 5 Kg/Kg of purified water and 0.38 Kg/Kg of L-cysteine were mixed and stirred to prepare a L-cysteine solution. At the temperature of 20˜30° C., the above organic phase was washed with (1) the L-cysteine solution and (2) 5 Kg/Kg of purified water and the palladium content in the organic phase was measured. At the temperature of 20˜30° C., after 0.1 Kg/Kg of activated charcoal was into the organic phase, the organic phase was stirred and filtered. After being washed with 1.8 Kg/Kg of ethyl acetate, the organic phases were combined and the palladium content was measured. If the palladium content was >7 ppm, the L-cysteine and activated charcoal removal operation was repeated until the palladium content was ≤7 ppm. The organic phase was then concentrated to 6˜8 vol. under reduced pressure at a temperature of no more than 50° C. After 7.9 Kg/Kg of methanol was into the organic phase, the organic phase was concentrated to 6˜8 vol. under reduced pressure at a temperature of no more than 50° C. This step was repeated once and the residual ethyl acetate in the organic phase was measured to make sure it was no more than 3%. After the organic phase was heated to 45˜55° C., 2 Kg/Kg of purified water was added dropwise to the organic phase at this the temperature while stirring, followed by adding 3 Kg/Kg of purified water dropwise to the organic phase at this temperature. The organic phase was then cooled down to 0˜10° C., maintained at this temperature, and stirred for crystallization. The mixture was filtered and eluted with a pre-cooled 0˜10° C. mixture of 0.79 Kg/Kg of methanol and 0.7 Kg/Kg of purified water. The solid thus obtained was dried in an oven at a temperature of no more than 60° C. After drying for 24 hours, samples were taken every 4-12 hours until loss on drying (LOD) is ≤1%. ¹H NMR (400 MHz, DMSO-d₆) δ 7.80-7.71 (m, 4H), 7.63 (d, J=6.1 Hz, 1H), 7.41 (td, J=7.8, 6.0 Hz, 1H), 7.31-7.21 (m, 2H), 7.03 (td, J=8.6, 2.6 Hz, 1H), 6.97-6.90 (m, 3H), 6.77 (d, J=9.7 Hz, 1H), 4.85 (p, J=6.7 Hz, 1H), 3.96 (dd, J=8.7, 5.4 Hz, 1H), 3.85 (dq, J=21.5, 6.8 Hz, 2H), 1.49 (d, J=6.9 Hz, 3H), 1.42 (s, 9H), 1.16 (d, J=6.4 Hz, 3H).

The experimental conditions and results of the conventional and inventive methods described above are summarized in Tables 5 and 6 below.

TABLE 5
Comparison of Conditions Between Conventional and Inventive Methods

| | | | | Compound of | | | |
|---|---|---|---|---|---|---|---|
| Synthetic | | Reaction | | Formula (3) | Compound of | Catalyst and | Reaction |
| Route | Solvent | Temp. | Base | Phosphate Salt | Formula (2) | Equivalent Amount | Time |
| Conventional | 1.4- | 85° C. | K₂CO₃ | 1.0 mol. eq. | 1.2 mol. eq. | Pd(dppf) Cl₂•CH₂Cl₂ | 3 h |
| Method | Dioxane/ | | 4.0 mol. eq. | | | 0.1 g/g | |
| | H₂O | | | | | | |
| Inventive | DMAC/ | 90° C. | K₃PO₄ | 1.0 mol. eq. | 1.10 mol. eq. | Triphenylphosphine | 3 h |
| Method | H₂O | | 1.5 mol. eq. | | | 0.008 mol. eq. | |
| | | | | | | Palladium acetate | |
| | | | | | | 0.004 mol. eq. | |

TABLE 6
Comparison of Experimental Results Between
Conventional and Inventive Methods

| Test item | Conventional Method | Inventive Method |
|---|---|---|
| Description | Pale yellow solid | White solid |
| Purity | 95% | 99% |
| Yield | 66% | 84% |

As shown in Tables 5 and 6 above, the inventive method surprisingly resulted in a product with a higher purity in a much higher yield than the conventional method even though a much smaller amount of a palladium catalyst was used. Without wishing to be bound by theory, it is believed that using Pd(dppf)Cl₂·CH₂Cl₂ in the conventional method is not cost-effective because a large amount (i.e., 0.1 g/g) of this catalyst is required, while only a small amount (i.e., 0.004 molar eq.) of palladium acetate is needed for the inventive method.

In addition, without wising to be bound by theory, it is believed that the column chromatography used in the conventional method is a relatively tedious process and lowers the yield of the final product. By contrast, although there are extra steps of removing the palladium catalyst in the inventive method, the solution extraction and recrystallization procedures used in the inventive method are relatively simple and can result in a product with a relatively high and consistent yield. In addition, unlike column chromatography, the solution extraction and recrystallization procedures used in the inventive method are readily scalable and suitable for commercial production.

### Example 4: Preparation 3-(4-((R)-2-aminopropoxy)phenyl)-N—((R)-1-(3-fluorophenyl)ethyl)imidazo[1,2-b]pyridazin-6-amine (the compound of formula (5))

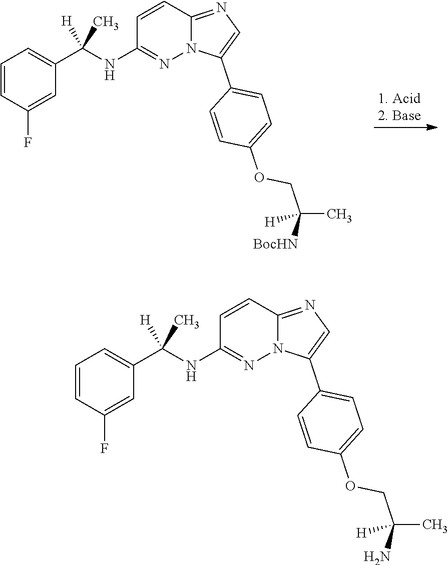 [SMILES: C[C@H](COc1ccc(-c2cnc3ccc(N[C@H](C)c4cccc(F)c4)nn23)cc1)NC(=O)OC(C)(C)C ; C[C@@H](N)COc1ccc(-c2cnc3ccc(N[C@H](C)c4cccc(F)c4)nn23)cc1]

3-(4-((R)-2-aminopropoxy)phenyl)-N—((R)-1-(3-fluorophenyl)ethyl)imidazo[1,2-b]pyridazin-6-amine (i.e., the compound of formula (5) described herein) was prepared using a conventional method and an inventive method as follows:

### Conventional Method

3-(4-((R)-2-aminopropoxy)phenyl)-N—((R)-1-(3-fluorophenyl)ethyl)imidazo[1,2-b]pyridazin-6-amine was prepared in a manner similar to the procedures described in step 3 of Example 21 in column 126 of U.S. Pat. No. 9,187,489. Specifically, 10 V of dichloromethane, 1.0 molar eq. of the compound obtained in Example 3 above, 2 V of trifluoroacetic acid were sequentially added into a four-neck flask. The reaction mixture was allowed to react at the temperature of 15˜25° C. for 1.5 hours. After IPC was qualified, the reaction mixture was washed with a sodium bicarbonate solution, and the aqueous phase was extracted with chloroform. The organic phases were combined, concentrate, and dried. The crude product was purified by column chromatography to obtain the title compound. The NMR of the compound thus obtained was consistent with the NMR of the product obtained from Step 3 in Example 21 in column 126 of U.S. U.S. Pat. No. 9,187,489.

### Inventive Method

5.84 Kg/Kg of anhydrous ethanol and 1.0 molar eq. of the compound obtained in Example 3 above were sequentially added into a four-neck flask. After the reaction mixture was heated to a temperature to 65˜75° C. and maintained at this temperature, 3 eq. of hydrochloric acid was added dropwise into the reaction mixture, which was allowed to react for 3 hours. After IPC was qualified, the temperature of the reaction mixture was maintained at 50˜70° C. and 6.66 Kg/Kg of purified water was added into the reaction mixture. While maintaining the temperature at 60˜70° C., a 10 N sodium hydroxide solution (including 0.735 Kg/Kg of purified water and 0.32 Kg/Kg of sodium hydroxide) was added dropwise into the reaction mixture. While maintaining the temperature at 60˜70° C., the reaction mixture was stirred for 1˜3 hours and the solid crystallization was checked. If there was no crystallization, a seed crystal suspension (which was prepared from 0.00075 Kg/Kg of seed crystals and 0.01 Kg/Kg of water) would be added into the reaction mixture, which would be stirred for another 1˜3 hours. If there was still no crystallization, the above operation would be repeated. The reaction mixture was then stirred for 1˜2 hours at 70˜75° C., cooled down to −5˜5° C., and maintained at this temperature for 3˜5 hours to complete crystallization. The reaction mixture was filtered and the filter cake was washed with an ethanol solution (1.85 Kg/Kg of purified water and 1.46 Kg/Kg of ethanol, −5˜5° C.). After 3.7 Kg/Kg of purified water was added to the filter cake, the mixture was stirred for 10˜30 minutes and filtered. The pH of the filter cake and content of ethylenediamine residue in the filter cake were measured. If the pH was greater than 8, the filter cake would be washed again with 3.7 Kg purified water until the pH of the filter cake was no more than 8.

The purity and the residual amount of palladium in the filter cake were measured. The criteria are as follows: purity ≥98.0%, the compound of formula (5) without fluorine (which is a major impurity)≤1.0%, other individual impurities≤0.30%, and palladium residue≤7 ppm. If the product did not meet any of the above criteria, the filtered cake was washed and filtered again while the temperature of the filter cake was maintained at a temperature of no more than 50° C., and the solid thus formed was dried in an oven. Samples were taken for measurement every 8˜12 hours until the residual KF was no more than 5%. The solid was collected and its weight was recorded as Y.

After adding 3.945 Y anhydrous ethanol into a reactor tank 1, the solid obtained above in an amount of Y was added to the reactor. The mixture was stirred at a temperature of 20˜30° C. to complete dissolution and was transferred to another reactor tank 2. 0.05 Y of activated charcoal was moistened with 0.1 Y˜0.2 Y of ethanol and added into tank 2 at 20˜30° C., and the mixture was stirred at 20˜30° C. for 3˜5 hours. The mixture was subsequently filtered, the filter cake was eluted with 1.578 Y ethanol, and the eluents were combined into tank 3. While maintaining the temperature of tank 3 at 30˜40° C., 7 Y of purified water was slowly added to the eluents and the mixture was stirred for 1˜3 hours. If there was no crystallization, the crystal seeding operation was performed (by adding 0.001 Y seed crystals and 0.01 Y water into the mixture to obtain a suspension and stirring the mixture for 1˜3 hours) and repeated if necessary. If there was solid precipitation, the mixture was cooled to −5˜5° C. and maintained at that temperature for 3˜5 hours to complete crystallization. The mixture was filtered and the filter cake was washed with an ethanol solution (which was prepared by mixing 2.5 Y of purified water and 1.97 Y of ethanol, stirred and maintained at −5˜5° C.). After the filter cake was added into SY of purified water, the mixture was stirred for 10˜30 min and filtered. A sample of the filter cake was taken to measure its purity or the content of residual palladium. If the purity was ≥98.0%, the compound of formula (5) without fluorine was ≤1.0%, any other individual impurity was ≤0.30%, and residual palladium was ≤7 ppm, the filter cake was collected and dried. If any of the above criteria was not met, repeat the above work-up operation until all of the above criteria were met. If the residual palladium met the relevant requirement, no activated charcoal was further used to remove palladium.

The solid obtained above was dried for 12 hours in an oven at a temperature of no more than 50° C. Samples were then taken every 8˜12 hours to check the residual solvents and water contents until the residual ethanol was ≤0.5%, the residual ethyl acetate was ≤0.5%, and the residual KF was ≤5%, 1H NMR (400 MHz, DMSO-d₆) δ 7.80-7.70 (m, 4H), 7.63 (d, J=6.1 Hz, 1H), 7.40 (td, J=7.9, 6.0 Hz, 1H), 7.31-7.21 (m, 2H), 7.07-6.99 (m, 1H), 6.96-6.89 (m, 2H), 6.77 (d, J=9.6 Hz, 1H), 4.84 (p, J=6.8 Hz, 1H), 3.85-3.69 (m, 2H), 3.17 (h, J=6.3 Hz, 1H), 1.59 (s, 2H), 1.48 (d, J=6.9 Hz, 3H), 1.09 (d, J=6.5 Hz, 3H).

The experimental conditions and results of the conventional and inventive methods described above are summarized in Tables 7 and 8 below.

TABLE 7
Comparison of Conditions Between Conventional and Inventive Methods

| Synthetic | | | | Compound of | |
|---|---|---|---|---|---|
| Route | Solvent | Reaction Temp. | Acid | Formula (4) | Reaction time |
| Conventional | CH₂Cl₂ | 15~25° C. | Trifluoroacetic | 1.0 molar eq. | 1.5 h |
| Method | | | 2 V | | |
| Inventive | EtOH | 65~75° C. | Conc. HCl | 1.0 molar eq. | 3 h |
| Method | | | 3.0 molar eq. | | |

TABLE 8
Comparison of Experimental Results Between
Conventional and Inventive Methods

| Test item | Conventional Method | Inventive Method |
|---|---|---|
| Description | White solid | White solid |
| Purity | 99.6% | 99.9% |
| Yield | 72% | 91% |

As shown in Tables 7 and 8 above, the inventive method surprisingly resulted in a product in a much higher yield than the conventional method. In addition, the yield in the conventional method (i.e., 72%) was much higher than the yield obtained from step 3 of Example 21 of U.S. Pat. No. 9,187,489 (i.e., about 29%). In other words, the yield of the inventive method was substantially higher than the yield obtained from step 3 of Example 21 of U.S. Pat. No. 9,187,489.

Without wishing to be bound by theory, it is believed that, although the reaction in the conventional method can be completed relatively quickly, the post-treatment column chromatography is a relatively tedious purification process, which resulted in a yield that is relatively low and inconsistent. By contrast, although the inventive method involved additional crystallization steps as a post-treatment of the manufacturing process, these steps are relatively simple to operate, and could achieve a high and consistent yield.

### Example 5: Preparation 3-(4-((R)-2-aminopropoxy)phenyl)-N—((R)-1-(3-fluorophenyl)ethyl)imidazo[1,2-b]pyridazin-6-amine (the Compound of Formula (5)) Adipate Salt

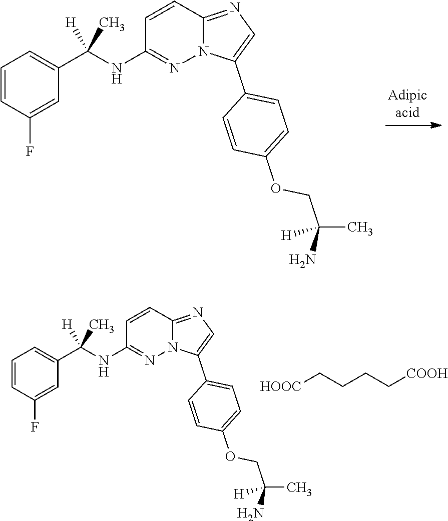 [SMILES: C[C@@H](N)COc1ccc(-c2cnc3ccc(N[C@H](C)c4cccc(F)c4)nn23)cc1 ; C[C@@H](N)COc1ccc(-c2cnc3ccc(N[C@H](C)c4cccc(F)c4)nn23)cc1 ; O=C(O)CCCCC(=O)O]

3-(4-((R)-2-aminopropoxy)phenyl)-N—((R)-1-(3-fluorophenyl)ethyl)imidazo[1,2-b]pyridazin-6-amine adipate salt (i.e., the adipate salt of the compound of formula (5) described herein) was prepared using a conventional method and an inventive method as follows:

### Conventional Method

3-(4-((R)-2-aminopropoxy)phenyl)-N—((R)-1-(3-fluorophenyl)ethyl)imidazo[1,2-b]pyridazin-6-amine adipate salt was prepared following the procedures described in Example 146 in columns 228-229 of U.S. Pat. No. 9,187,489. Specifically, 10 V of 1-propanol, 1.0 molar eq. of the compound of formula (5) obtained from Example 4 above, 1.1 molar eq. of adipic acid were sequentially added into a four-neck flask. The reaction mixture was stirred at 40° C. for 24 hours, cooled down to 20° C., and stirred for another 0.5 hour to undergo crystallization. The reaction mixture was filtered and the solid thus obtained was dried to give the title compound. The NMR of the compound thus obtained was consistent with the NMR of the product obtained from Step 3 in Example 21 in column 126 of U.S. Pat. No. 9,187,489.

### Inventive Method

3.156 Kg/Kg of anhydrous ethanol and 1.0 molar equivalent of the compound of formula (5) obtained from Example 4 above were added into a reactor. The reaction mixture was heated to 45-55° C. (target: 50° C.) and stirred until a clear solution was formed. While maintaining the temperature at 10-30° C. (target: 20° C.), an adipic acid solution was prepared by dissolving 0.396 Kg/Kg of (1.1 molar equivalent) adipic acid into a mixed solution of 2.367 Kg/Kg of anhydrous ethanol and 3.0 Kg/Kg of purified water. While maintaining the temperature at 45-55° C. (target: 50° C.), the adipic acid solution was added into the reactor to form a mixture, which was stirred for 1-3 hours (target: 2 hours) after the addition was complete. The reaction mixture was then concentrated to 4.0-6.0 volume (target: 5.0 volume) under reduced pressure at 45-55° C. (target: 50° C.), and stirred at this temperature for 0.5-1.5 hours (target: 1 hour) until there was solid precipitation. After the mixture was cooled down to 20-30° C. (target: 25° C.) at a cooling speed of 5-15° C./h (target: 10° C./h), 8.0 Kg/Kg of purified water was added to the mixture at 20-30° C. (target: 25° C.) and the mixture was stirred at this temperature for 0.5-1.5 hours (target: 1 hour). The mixture was then cooled down to −5˜5° C. (target: 0° C.) at a cooling speed of 5-15° C./h (target: 10° C./h), maintained for 4˜8 h (target: 6 h) at this temperature, and filtered. The solid thus obtained was dried at a temperature of no more than 70° C. until LOD was not more than 5%. The solid was then collected to give a crude product.

The crude product was purified and milled as follows: After 4.02 Kg/Kg of anhydrous ethanol and 2.2 Kg/Kg of purified water were added into a reactor and stirred, the crude product obtained above was added to the mixture. The reaction mixture was heated to 65-70° C. and stirred until a clear solution was obtained. The recommended stirring time and rotation speed were 0.5-1 hour and 60-100 rpm, respectively. The reaction mixture was then cooled to 50-55° C. (target temperature: 53° C.) with a recommended cooling rate of 5-10° C./h. After 0.01 Kg/Kg of seed crystals of the compound of formula (5) adipate salt were added to the mixture at 50˜55° C., the mixture was stirred for 1˜3 hours (target: 2 hours) at this temperature. The reaction mixture was cooled down to 20-30° C. (target temperature: 25° C.) with recommended cooling rate of 5-10° C./h. After 8.4 Kg/Kg of purified water was slowly added to the mixture at 20-30° C. in 1-3 hours (target: 2 hours), the mixture was stirred for 1˜3 hours (target: 2 hours) at this temperature. The mixture was cooled to −5 to 5° C. (target temperature: 0° C.) at a cooling rate of 5 to 10° C./h, and stirred at this temperature for 4 to 8 hours (target: 6 hours). While maintaining the temperature at −5 to 5° C. (target temperature: 0° C.), the reaction mixture was passed through a wet mill (Blade no. 2, 6, and 6; rotation speed: 6,000-10,000 rpm, target: 8,000 rpm). Samples were taken every 20-30 minutes to measure the particle size of the solids after wet milling until D50 was no more than 45 μm (target: 35-45 μm). The reaction mixture was filtered through a filter with filter dryer sheath pre-cooled to T=−5˜5° C. The filter cake was washed with a mixture of 1.1 Kg/Kg of purified water and 0.39 Kg/Kg of anhydrous ethanol, which was pre-cooled to −5˜5° C. and maintain for at least 0.5 hour. The solid thus obtained was dried at 50˜60° C. in an oven under a controlled temperature of no more than 70° C. for 12 hours. Samples were taken every 4˜8 hours to track the residual ethanol and make sure the content of residual ethanol was no more than 0.4%. After drying, the solid was cooled to 20˜30° C. to give the title compound in high purity. ¹H NMR (400 MHz, DMSO-d₆) δ 8.97 (s, 4H), 7.79-7.64 (m, 5H), 7.37 (td, J=7.9, 6.0 Hz, 1H), 7.30-7.18 (m, 2H), 7.04-6.91 (m, 3H), 6.80 (d, J=9.7 Hz, 1H), 4.83 (p, J=6.7 Hz, 1H), 4.10-3.89 (m, 2H), 3.52-3.35 (m, 1H), 2.14 (h, J=3.4 Hz, 4H), 1.50 (h, J=3.7, 3.3 Hz, 4H), 1.46 (d, J=6.9 Hz, 3H), 1.25 (d, J=6.6 Hz, 3H). The NMR of the compound thus obtained was consistent with the NMR of the product obtained from Step 3 in Example 21 in column 126 of U.S. Pat. No. 9,187,489. The solid powder thus obtained had a particle size D10 of 7.2 μm, a particle size D50 of 44 μm, and a particle size D90 of 107 μm.

The experimental conditions and results of the conventional and inventive methods described above are summarized in Tables 9 and 10 below.

TABLE 9
Comparison of Conditions Between Conventional and Inventive Methods

| Synthetic | | | | | Compound of | | |
|---|---|---|---|---|---|---|---|
| Route | Solvent | Reaction Temp. | | Adipic acid | Formula (5) | Reaction time | |
| Conventional | 1-Propanol | 40° | C. | 1.1 mol. eq. | 1.0 mol. eq. | 24 | h |
| Method | | | | | | | |
| Inventive | EtOH | 45~55° | C. | 1.1 mol. eq. | 1.0 mol. eq. | 1-3 | hours |
| Method | | | | | | | |

TABLE 10
Comparison of Experimental Results Between
Conventional and Inventive Methods

| Test item | Conventional Method | Inventive Method |
|---|---|---|
| Description | White solid | White solid |
| Purity | 99.6% | 99.7% |
| Yield | 91% | 94% |

As shown in Tables 9 and 10 above, the inventive method surprisingly resulted in a product with a yield higher than the conventional method even though the inventive method involved more steps. In addition, without wishing to be bound by theory, it is believed that the inventive method used heating and cooling during the crystallization process, which can result in a product with a more uniformed particle size. In addition, the inventive method included a wet milling step, which can result in a product having a desired particle size.

### Example 6: Dissolution measurements of 3-(4-((R)-2-aminopropoxy)phenyl)-N—((R)-1-(3-fluorophenyl)ethyl)imidazo[1,2-b]pyridazin-6-amine monoadipate (the monoadipate salt of the compound of formula (5))

Particles containing 3-(4-((R)-2-aminopropoxy)phenyl)-N—((R)-1-(3-fluorophenyl)ethyl)imidazo[1,2-b]pyridazin-6-amine monoadipate having eight different size distributions (i.e., Batch Nos. 1-8) were prepared either without milling or by using a wet milling process similar to that described in Example 5. The particles size distributions of these eight different batches are summarized in Table 11 below.

TABLE 11

| | Particle Size Distribution | | |
|---|---|---|---|
| | D10 (μm) | D50 (μm) | D90 (μm) |
| Batch No. 1 | 1.6 | 6.4 | 78 |
| Batch No. 2 | 1.9 | 43 | 129 |
| Batch No. 3 | 7.2 | 44 | 107 |
| Batch No. 4 | 9.0 | 45 | 91 |
| Batch No. 5 | 13 | 48 | 90 |
| Batch No. 6 | 8.1 | 44 | 93 |
| Batch No. 7 | 23 | 64 | 123 |
| Batch No. 8 | 33 | 90 | 167 |

Capsules containing the particles in the above eight batches were prepared. The dissolution amounts of these capsules were measured by a Distek Model 2500 RTD Dissolution System available from Distek, Inc. (North Brunswick, NJ) using the dissolution parameters summarized in Table 12 below.

TABLE 12
Dissolution Parameters

| | |
|---|---|
| Method | II (Paddles) with capsule sinkers |
| Sample size | 6 capsules |
| Rotation speed | 50 rpm/min |
| Dissolution medium | 0.05 mol/L acetic acid-sodium acetate buffer |
| | solution, pH 4.0 |
| Dissolution volume | 900 mL |
| Medium | 37 ± 0.5° C. |
| temperature | |
| Sampling volume | Dissolution: 20 mL |
| | Dissolution profile: 20 mL |
| Media replacement | Dissolution: No media replacement |
| | Dissolution profile: 20 mL |
| Sampling time | Dissolution: 45 min |
| | Dissolution profile: 5 min, 10 min, 15 min, 20 min, |
| | 30 min, 45 min, 60 min (and an additional 15 |
| | minutes at 250 rpm) |
| Sampling method | Manual Sampling |
| Discard the first | 10 mL |
| filtrate | |
| Filter | PVDF filter of 0.45 μm pore size (25 mm GD/X |
| | syringe filter, Whatman Ltd. or equivalent) |

The dissolution amounts of the active ingredient from the capsules were measured by ACQUITY Arc HPLC System with a 2998 PDA detector and a 2489 UV/Vis detector available from Waters Corporation (Milford, MA) using the conditions summarized in Table 13 below.

TABLE 13
Chromatographic Conditions

| | |
|---|---|
| Column | Ascentis Express C18 (4.6 mm ID × 100 mm, 2.7 |
| | μm, SIGMA-ALDRICH) |
| Flow rate | 2.2 mL/minute |
| Column temperature | 40° C. |
| Injection volume | 5 μL |
| Measurement time | 4 minutes |
| Mobile phase | 0.02 mol/L NaClO₄-0.1% H₃PO₄ in water:ACN = |
| | 3:1(v/v) |
| Detection | 256 nm |
| wavelength | |
| Needle Wash | Water:ACN = 1:1(v/v) |

During use, the HPLC was equilibrated until a flat baseline was obtained. The chromatographs of sample solutions was obtained using the minimum suggested injection sequence outlined in Table 14. The diluent was prepared by mixing 500 mL of 0.05 mol/L acetic acid-sodium acetate buffer solution (pH 4.0) with 500 mL of acetonitrile. Standard solutions 1 and 2 were prepared by the following procedures: After 28 mg (±2.8 mg) of the monoadipate salt of the compound of formula (5) as a reference standard was accurately weighed in a 20 mL volumetric flask, approximately 15 mL of the diluent obtained above was added to the volumetric flask. The mixture was sonicated with occasional shaking until the solid was completely dissolved. The solution was allowed to cool to temperature, diluted to 20 mL with the above diluent, and mixed thoroughly. 2 mL of the above solution was accurately pipetted into a 20 mL volumetric flask, diluted to 20 mL with the dissolution medium, and mix thoroughly.

TABLE 14

| Order of Sequence | Number of injections |
|---|---|
| Blank (Diluent) | At least 1 |
| Standard solution 1 | 5 |
| Standard solution 2 | 2 |
| Each Sample solution | 1 |
| BKT Standard Solution 1 (No more than 10 | 1 |
| sample injections and at the end of sequence) | |

The HPLC needs to meet the following requirements to be suitable to measure the dissolution amounts of the active ingredient: (1) no significant interference peak should be observed in the Blank chromatogram at the retention time of interest; (2) the relative standard deviation of the peak area (xᵢ) for the active ingredient converted by weight in the first seven injections of Standard Solution (5×Std1+2×Std2) should be not more than 2.0%; and (3) the peak area (xᵢ) for the active ingredient converted by weight in BKT Standard Solution 1 (Std 1 injected after each 10-sample injections and at the end of sequence) must be within 98.0˜102.0% of average peak area ratio (R_{Ave}) in the first seven injections of Standard Solutions.

The dissolution amounts of the active ingredient (i.e., the compound of formula (5) monoadipate salt) were calculated using the following four equations:

$${X}_{i} = \frac{{A}_{Si}}{{W}_{Si} \times {F}_{p}} {R}_{Ave} = \frac{\sum {X}_{i}}{7} \\ Dissolution Amount ( \% ) = \frac{405.47 \times 900 \times {A}_{T}}{{R}_{Ave} \times 551.61 \times 200 \times L} \times 100 \\ Accumulative Dissolution Amount at each time point ( \% ) = {A}_{n} + \frac{( {A}_{1} + {A}_{2} + … + {A}_{n - 1} ) \times {V}_{1}}{900}$$

in which A_{Si} is the peak area of the active ingredient in Standard Solution; W_{Si} is the weight of reference standard of the active ingredient (mg); Fₚ is the purity factor of the reference standard of the active ingredient; xᵢ is the peak area ratio of the active ingredient in Standard Solution; R_{Ave} is the average peak area ratio of the active ingredient in the first seven injections of Standard Solution; 405.47 is the molecular weight of the free form of the active ingredient (i.e., the compound of formula (5)); 551.61 is the molecular weight of the active ingredient; A_{T} is the peak area of the active ingredient obtained from Sample Solution; L is the labeled amount of a capsule (mg); Aₙ is the dissolution amount at each time point; and V₁ is the constant sampling volume at each time point.

The dissolution results are summarized in Table 15 below.

TABLE 15

| | | Time point (min) | | | | | | |
|---|---|---|---|---|---|---|---|---|
| | | 0 | 10* | 15 | 20 | 30 | 45 | 60 |
| Batch No. 1 | Dissolution Amount (%) | 0 | 26 | 80 | 95 | 98 | 100 | 100 |
| | RSD (%) | 0 | 47.8 | 8.9 | 3.2 | 2.3 | 1.8 | 1.7 |
| Batch No. 2 | Dissolution Amount (%) | 0 | 45 | 80 | 88 | 92 | 94 | 96 |
| | RSD (%) | 0 | 27.7 | 4.8 | 3.8 | 3.6 | 3.2 | 2.9 |
| Batch No. 3 | Dissolution Amount (%) | 0 | 11 | 71 | 89 | 97 | 100 | 102 |
| | RSD (%) | 0 | 134.9 | 14.9 | 5.2 | 3.1 | 2.5 | 2 |
| Batch No. 4 | Dissolution Amount (%) | 0 | 17 | 72 | 88 | 95 | 97 | 98 |
| | RSD (%) | 0 | 71 | 24.2 | 9 | 3.6 | 1.8 | 0.6 |
| Batch No. 5 | Dissolution Amount (%) | 0 | 24 | 76 | 88 | 94 | 97 | 98 |
| | RSD (%) | 0 | 104 | 12.5 | 5.2 | 2.7 | 2.4 | 2 |
| Batch No. 6 | Dissolution Amount (%) | 0 | 19 | 68 | 84 | 91 | 96 | 98 |
| | RSD (%) | 0 | 128.8 | 22.5 | 13.4 | 6.9 | 3.1 | 1.9 |
| Batch No. 7 | Dissolution Amount (%) | 0 | 52 | 80 | 88 | 96 | 100 | 101 |
| | RSD (%) | 0 | 55.9 | 11.4 | 6.7 | 3.7 | 2.2 | 1.6 |
| Batch No. 8 | Dissolution Amount (%) | 0 | 2 | 30 | 46 | 60 | 72 | 80 |
| | RSD (%) | 0 | 125.3 | 14.3 | 4.2 | 3.8 | 3.9 | 3.6 |

*The RSD values for 10-minute time point are relatively large due to the fact that tested capsules were not completely dissolved.

As shown in Tables 11 and 15, Batch No. 8 (which contained particles having a particle size D50 of 90 μm) exhibited a dissolution amount of 72 wt % in 45 minutes, which is below the relevant regulatory requirements (e.g., a dissolution amount of 75 wt % or 80 wt % in 45 minutes). By contrast, Batch Nos. 1-7 (which contained particles having a particle size D50 of 6.4-64 μm) were able to meet the relevant regulatory requirements.

In addition, the product yield of Batch No. 1 (which contained particles having a particle size D50 of 6.4 μm) is about 67%, which is lower than the product yields of Batch Nos. 2-8 (e.g., from 86-96%). Thus, although the product yield of Batch No. 1 is acceptable, Batch No. 1 is less preferable than Batch Nos. 2-8.

Other embodiments are within the scope of the following claims.

What is claimed is:

1. A manufacturing method, comprising:
reacting a compound of formula (2): 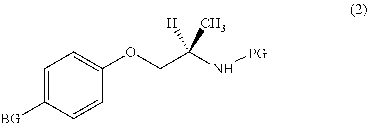
with a salt of a compound of formula (3): 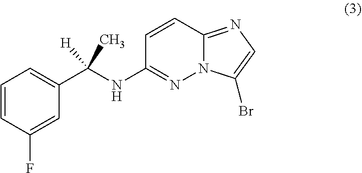 [SMILES: C[C@@H](Nc1ccc2ncc(Br)n2n1)c1cccc(F)c1]
in the presence of a palladium catalyst, a base, and a solvent to form a compound of formula (4): 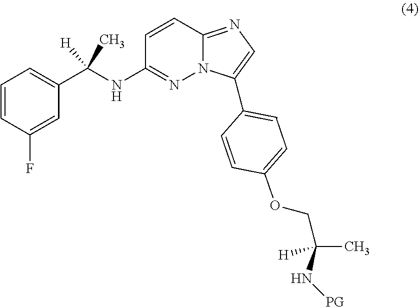
wherein BG is a boronic ester or boronic acid group, and PG is a protecting group for a nitrogen atom.

2. The method of claim 1, wherein further comprising removing the protecting group PG from the compound of formula (4) to form a compound of formula (5):

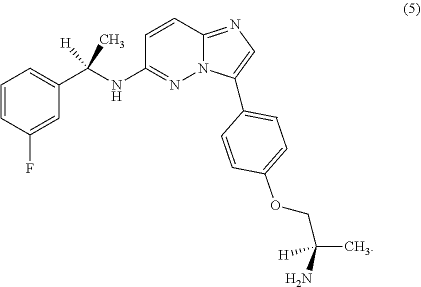 [SMILES: Cc1cc(OC[C@@H](C)N)ccc1-c1cnc2ccc(N[C@H](C)c3cccc(F)c3)nn12]

3. The method of claim 3, further comprising reacting the compound of formula (5) with an adipic acid to form an adipate salt of the compound of formula (5).

4. The method of claim 1, wherein the salt of the compound of formula (3) is a phosphate salt.

5. The method of claim 4, wherein the phosphate salt of the compound of formula (3) comprises about 1.5 molar of phosphoric acid per 1 molar of the compound of formula (3).

6. The method of claim 1, wherein PG is a tert-butoxycarbonyl group, a fluorenylmethoxycarbonyl group, or a benzyloxycarbonyl group.

7. The method of claim 1, wherein BG is

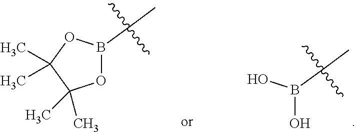

8. The method of claim 1, wherein the palladium catalyst comprises a reaction product of a monodentate phosphine or a bidentate phosphine with a palladium compound.

9. The method of claim 8, wherein the monodentate phosphine is triphenylphosphine, tri-t-butylphosphine, or tris(2-methylphenyl)phosphine.

10. The method of claim 8, wherein the bidentate phosphine is 1,1-bis(diphenylphosphino)methane or 1,2-bis(diphenylphosphino)ethane.

11. The method of claim 8, wherein the palladium compound is palladium chloride or palladium acetate.

12. The method of claim 8, wherein the palladium catalyst comprises a reaction product of palladium acetate and triphenylphosphine.

13. The method of claim 1, wherein the palladium catalyst is from about 0.1 mol % to about 5 mol % based on the amount of the compound of formula (3).

14. The method of claim 1, wherein the base comprises potassium hydroxide, sodium hydroxide, sodium carbonate, potassium carbonate, or cesium carbonate.

15. The method of claim 1, wherein the solvent comprises dimethylacetamide, dimethylformamide, N-methyl-2-pyrrolidone, dimethyl sulfoxide, 4-dioxane, or diethylene glycol dimethyl ether.

16. The method of claim 1, further comprising reacting a compound of formula (1): 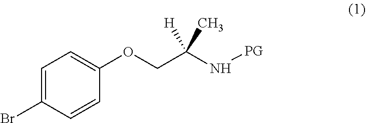
with a boron-containing agent to form the compound of formula (2).

17. The method of claim 16, wherein the boron-containing agent is bis(pinacolato)diboron.

18. The method of claim 16, further comprising reacting 1-bromo-4-fluorobenzene with D-alaninol to form (R)-1-(4-bromophenoxy)propan-2-amine, and protecting the amino group in (R)-1-(4-bromophenoxy)propan-2-amine to form the compound of formula (1).

19. The method of claim 18, wherein protecting the amino group in (R)-1-(4-bromophenoxy)propan-2-amine is performed by reacting (R)-1-(4-bromophenoxy)propan-2-amine with di-tert-butyl dicarbonate.

20. The method of claim 1, further comprising reacting a compound of formula (6): 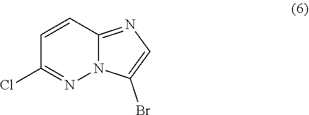 [SMILES: Clc1ccc2ncc(Br)n2n1]
with a compound of formula (7): 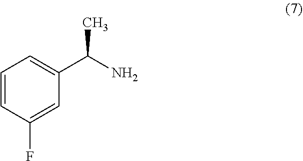
to form the compound of formula (3).

21. The method of claim 20, further comprising reacting the compound of formula (3) with an acid to form the salt of the compound of formula (3).

22. A pharmaceutical composition, comprising:
particles comprising 3-{4-[(2R)-2-aminopropoxy]phenyl}-N-[(1R)-1-(3-fluorophenyl)ethyl]-imidazo[1,2-b]pyridazin-6-amine monoadipate; and
a pharmaceutically acceptable carrier;
wherein the particles have a particle size D50 of from about 20 μm and 70 μm.

23. The composition of claim 22, wherein the particles have a particle size D50 of from about 20 μm and 60 μm.

24. The composition of claim 22, wherein the particles have a particle size D50 of from about 25 μm and 55 μm.

25. The composition of claim 22, wherein the particles have a particle size D90 of from about 50 μm and 150 μm.

26. The composition of claim 22, wherein the particles have a particle size D10 of from about 1 μm and 25 μm.

27. The composition of claim 22, wherein the composition is a capsule or a tablet.
